(19) United States
(12) Patent Application Publication
(10) Pub. No.: US 2014/0024975 A1
(43) Pub. Date: Jan. 23, 2014

(54) HIFU COMPONENTS WITH INTEGRATED CALIBRATION PARAMETERS

(71) Applicants: Blake Little, Bothell, WA (US); Tanar Ulric, Woodinville, WA (US)

(72) Inventors: Blake Little, Bothell, WA (US); Tanar Ulric, Woodinville, WA (US)

(73) Assignee: LIPOSONIX, INC., Hayward, CA (US)

(21) Appl. No.: 13/551,299

(22) Filed: Jul. 17, 2012

(51) Int. Cl. A61N 7/02

(52) U.S. Cl. 601/3

(57) ABSTRACT

Devices, systems, and methods for increasing the accuracy of ultrasound power delivered to and received from a patient by an HIFU system having exchangeable components. The system components include one or more memory devices in which calibration parameters for the component are stored, such as when the component is manufactured. During operation of the system, the calibration parameter data is received from the memory and used by the system to adjust the output power of one or more ultrasound sources and/or received signal levels based on the received calibration parameters. The calibration parameters may include data relating to impedance, sensitivity, transfer functions, linearity, or other data that could affect the power delivered to a patient by the component.

### TECHNICAL FIELD

The present invention relates generally to medical ultrasound systems and, more particularly, to systems and methods of improving the accuracy with which ultrasound power is delivered to a patient by medical ultrasound systems having interchangeable components.

### BACKGROUND

Ultrasound is commonly known for its uses in non-therapeutic medical procedures, such as tissue imaging for diagnostic purposes. High Intensity Focused Ultrasound (HIFU) is a medical procedure that uses highly focused ultrasound energy to heat and destroy diseased or other unwanted tissue. HIFU systems thereby use ultrasound energy to provide therapeutic benefits in a non-invasive manner. In order to achieve the desired physical effects in the targeted tissue, HIFU involves much higher power levels than diagnostic ultrasound. However, the rate at which acoustic energy is delivered to the patient must be balanced between the amount of power needed to achieve the desired effect and a desire to avoid damaging tissue outside of the targeted area. This is particularly true in aesthetic medicine, where there is less tolerance for surrounding tissue damage by practitioners and their clientele than is typically the case for other types of medicine. Therefore, it is desirable to maintain tight control over the amount of power delivered to the patient by HIFU systems so that the effectiveness of treatment may be optimized.

An HIFU system typically includes a source of acoustic energy coupled to a transducer that may be part of a handpiece. The handpiece is typically attached to the acoustic energy source by a cable that allows the handpiece to be easily positioned on a patient by the treating physician. Because the impedances and other electrical and acoustic characteristics of the acoustic energy source, cable, and transducer tend to vary from unit to unit, HIFU systems are typically tuned or calibrated at the factory as a matched set. However, while tuning each system at the factory may improve the accuracy with which ultrasound energy is delivered to the patient, tuning also increases the cost of production. In addition, factory calibration means that the source, cable, and transducer become a matched set that cannot be interchanged with other systems without a re-calibration or loss of power control accuracy. That is, a system would need to be re-calibrated each time a source, cable, or transducer was exchanged with another system. Moreover, a re-calibration would also be required whenever a user wished to add a new or updated handpiece that provides new capabilities. Calibration in the field would typically require test equipment and user training, adding significant expense to the cost of operating HIFU systems. Returning systems to the factory for calibration each time a new component is attached to the system is also undesirable due to the cost of shipping and lost availability of the system. These costs are particularly onerous for systems where the transducer is part of a consumable component that is regularly replaced, such as a Replicable Treatment Cartridge (RTC).

Thus, there is a need for improved devices, systems, and methods for accurately delivering ultrasound energy to a patient using HIFU systems that allow components to be exchanged without the need for re-calibration.

### BRIEF SUMMARY

In one embodiment, a method is provided for controlling the acoustic power output of an ultrasound treatment system. The method includes retrieving a calibration parameter of a component removably coupled to the ultrasound treatment system from a memory associated with the component. The method further includes adjusting an output signal of an ultrasound signal transmitter based at least in part on the retrieved calibration parameter of the component.

In another embodiment, a replaceable treatment cartridge for an ultrasound treatment system is provided that includes an acoustic transducer and a memory. The memory is configured to store data relating to a calibration parameter of the replaceable treatment cartridge.

In another embodiment, an ultrasound treatment system is provided that includes an ultrasound signal transmitter and a signal port coupled to the ultrasound signal transmitter that is configured to accept a treatment head. The treatment system further includes a processor configured to obtain data relating to a calibration parameter of a component of the ultrasound treatment system, and to determine an output level of the ultrasound signal transmitter based at least in part on the data relating to the calibration parameter of the component.

### BRIEF DESCRIPTION OF THE SEVERAL VIEWS OF THE DRAWINGS

The accompanying drawings, which are incorporated in and constitute a part of this specification, illustrate various embodiments of the invention and, together with the general description of the invention given above and the detailed description of the embodiments given below, serve to explain the embodiments of the invention.

FIG. 1 is a diagrammatic view of a High Intensity Focused Ultrasound (HIFU) system including a console, treatment head, and a Replaceable Treatment Cartridge (RTC).

FIG. 2 is a schematic of the HIFU system in FIG. 1.

FIG. 3 is a diagram of a Z-parameter model of the HIFU system in FIG. 2.

FIG. 4 is a diagram illustrating test measurement set up for measuring a transmitter output impedance of the console in FIG. 2.

### DETAILED DESCRIPTION

Embodiments of the invention are generally related to High Intensity Focused Ultrasound (HIFU) systems and components that provide a system controller with information relating to performance characteristics of the components. Embodiments also include methods for improving the accuracy with which ultrasound energy is delivered to a patient by an HIFU system based on the provided performance characteristics. To this end, the HIFU system components may include one or more memory devices that store data relating to an impedance, sensitivity, transfer function, linearity, correction factor, frequency response, or any other calibration parameter that could affect the power delivered to a patient by the component. The data may represent calibration parameters obtained using test equipment and stored in the component memory at a production facility, as well as calibration parameters determined by the HIFU system in the field. When the component is attached to the system, calibration parameter data may be downloaded from the memory. The calibration parameter data may then be used by the system to adjust the electrical output power of one or more ultrasound sources and/or adjust monitored ultrasound signal levels. By obtaining transmission characteristics associated with a component from the memory device in the component, the system may adjust power output levels to compensate for performance characteristics specific to that component. The system may thereby maintain a more consistent power delivery level at the patient as compared to systems lacking this feature.

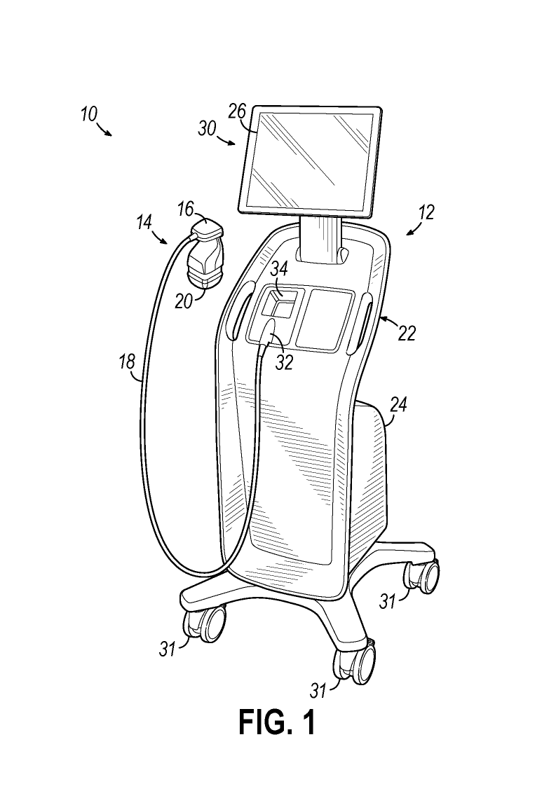

With reference to FIG. 1, an HIFU system 10 includes a console 12, a treatment head 14 that includes a hand-piece 16 that couples to the console 12 via a cable 18, and a Replaceable Treatment Cartridge (RTC) 20 that is removably coupled to the treatment head 14. The console 12 may include a base unit 22 comprising a housing 24 that provides space for system circuitry and serves as a platform for the system 10 and a display 26.

In an embodiment of the invention, the display 26 may be a touch screen device which provides a user interface 30 that allows an operator to control the system 10. In alternative embodiments of the invention, other means of viewing and entering data may be provided, in which case the user interface 30 may include separate display and data entry devices, such as a video monitor and keypad (not shown). Embodiments are therefore not limited to a particular type of user interface 30. For example, in addition to the display 26 discussed above, the user interface 30 may include additional output devices, such as alphanumeric displays, a speaker, and other audio and visual indicators. The user interface 30 may also include additional input devices and controls such as an alphanumeric keyboard, a pointing device, keypads, pushbuttons, control knobs, microphones, etc., capable of accepting commands or input from the operator and transmitting the entered input to the HIFU system 10.

A plurality of wheels 31 may be coupled to the base 20 so that the system can be rolled between areas where patients are treated. A connection point 32 having one or more signal ports and that is configured to accept a connectorized end of cable 18 may be provided in the base unit 22 to facilitate coupling different treatment heads 14 to the console 12. The base unit 22 may also include one or more receptacles 34 that provide a convenient storage place for system components and accessories, such as the hand-piece 16.

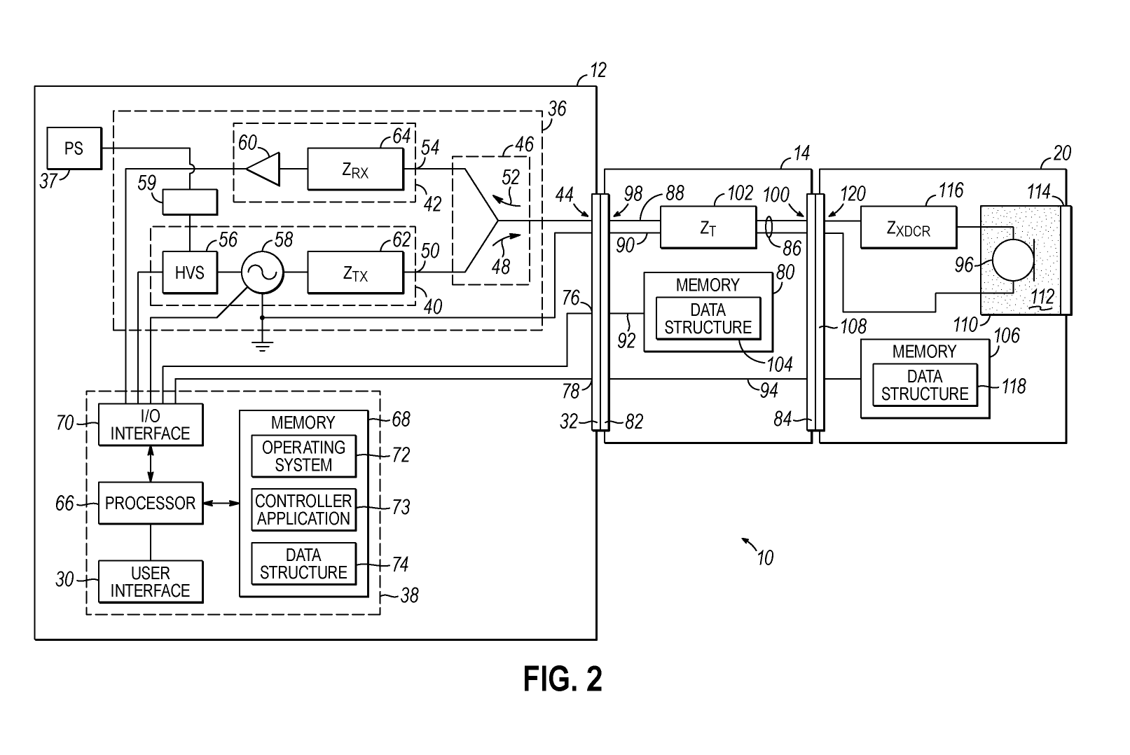

FIG. 2 is a block diagram of the HIFU system 10 that illustrates functional aspects of the console 12, treatment head 14, and RTC 20 in accordance with embodiments of the invention. The console 12 includes an ultrasound transceiver 36, a power supply 37, and a controller 38. The ultrasound transceiver 36 includes an ultrasound signal transmitter 40 and an ultrasound signal receiver 42 that are each operatively coupled to an ultrasound signal port 44 via a coupling element 46. The ultrasound signal port 44 is typically part of the connection point 32, and may be comprised of a coaxial cable, twisted pair, or other single or multi-conductor connection. The coupling element 46 may be a directional coupler, a switch, or any other suitable device that couples an ultrasound transmit signal 48 from an output 50 of transmitter 40 to the signal port 44, and that couples an input signal 52 from the signal port 44 to an input 54 of receiver 42. For example, the coupling element 46 may simply be a common point of connection, with the input of the receiver and/or the output of the transmitter gated so that the receiver 42 is de-coupled from the signal port 44 when the signal transmitter 40 is outputting the transmit signal 48.

The transmitter 40 includes a High Voltage Supply (HVS) 56 that provides a DC voltage to an ultrasound driver 58 that is selectively activated by the controller 38. The HVS 56 may be coupled to the console power supply 37 through a monitor circuit 59, and may cooperate with the ultrasound driver 58 to selectively generate the ultrasound transmit signal 48 at an adjustable power level that is sufficient to provide therapeutic benefits when provided to a patient via the RTC 20. To this end, the HVS 56 may have an adjustable output voltage level, and the ultrasound driver 58 may be selectively activated by the controller 38. The HVS monitor circuit 59 may include current and/or voltage monitor circuits that are configured to provide a signals to the controller 38 relating to one or more current and/or voltage levels in or provided by the HVS 56.

The receiver 42 may include an input amplifier 60 configured to amplify or otherwise process the received ultrasound signal 52 so that the received signal is suitable for processing by the controller 38. In any case, the transmitter 40 has a characteristic electrical output impedance represented by Z_{TX} 62, and the receiver 42 has a characteristic electrical input impedance represented by Z_{RX} 64. Although illustrated as having a single ultrasound transceiver 36, embodiments of the invention are not limited to a particular number of ultrasound transceivers 36. For example, system 10 may have a plurality of (e.g., 16) ultrasound transceivers 36, with each transceiver being coupled to one of a plurality of ultrasound signal ports 44. The system 10 may also have a different number of transmitters 40 than receivers 42—i.e., one or more of a plurality of transceivers 36 may include just the transmitter 40 or just the receiver 42.

The controller 38 includes the user interface 30, a processor 66, a memory 68, and an input/output (I/O) interface 70. The user interface 30 may be operatively coupled to the processor 66 of controller 38 in a known manner to allow a system operator to interact with the controller 38. The processor 66 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on and/or controlled by operational instructions that are stored in the memory 68. Memory 68 may be a single memory device or a plurality of memory devices including but not limited to read-only memory (ROM), random access memory (RAM), volatile memory, non-volatile memory, static random access memory (SRAM), dynamic random access memory (DRAM), flash memory, cache memory, or any other device capable of storing digital information. Memory 68 may also include a mass storage device (not shown) such as a hard drive, optical drive, tape drive, non-volatile solid state device or any other device capable of storing digital information.

Processor 66 may operate under the control of an operating system 72 that resides in memory 68. The operating system 72 may manage controller resources so that computer program code embodied as one or more computer software applications, such as a controller application 73 residing in memory 68 may have instructions executed by the processor 66. In an alternative embodiment, the processor 66 may execute the applications 73 directly, in which case the operating system 72 may be omitted. One or more data structures 74 may also reside in memory 68, and may be used by the processor 66, operating system 72, and/or controller application 73 to store data, such as system calibration parameter values. The I/O interface 70 operatively couples the processor 66 to other components of the HIFU system 10, including the HVS 56, the ultrasound driver 58, the ultrasound signal receiver 42, a first data port 76 and a second data port 78. The data ports 76, 78 may be comprised of pins or other suitable coupling elements included in the connection point 32. The I/O interface 70 may include signal processing circuits that condition incoming and outgoing signals so that the signals are compatible with both the processor 66 and the components to which the processor 66 is coupled. To this end, the I/O interface 70 may include analog to digital (A/D) and/or digital to analog (D/A) converters, voltage level and/or frequency shifting circuits, optical isolation and/or driver circuits, and/or any other analog or digital circuitry suitable for coupling the processor 66 to the other components of the HIFU system 10.

The treatment head 14 includes a memory 80 and cable 18. The cable 18, in turn, may be terminated by a console connector 82 at a proximal end and by an RTC connector 84 in the hand-piece 16 at a distal end. The cable 18 may further include one or more transmission lines 86 each comprised of one or more conductors 88, 90, as well as a first data channel 92 and a second data channel 94. Typically, the number of transmission lines 86 in cable 18 will correspond to the number of ultrasound transceivers 36 and/or a number of transducer elements comprising a transducer 96 in the RTC 20, with each transceiver 36 being coupled to a corresponding transducer element by a dedicated transmission line 86. To this end, the console connector 82 may include one or more ultrasound signal ports 98 that couple to one or more ultrasound signal ports 44 of connection point 32. A corresponding number of ultrasound signal ports 100 in the RTC connector 84 may be coupled to the distal end of transmission line 86 so that when the console connector 82 is coupled to the connection point 32, the ultrasound transceiver 36 is coupled to the ultrasound signal port 100 of RTC connector 84, and thereon to the transducer 96.

The first and second data channels 92, 94 may each be comprised of one or more conductors (not shown) that provide one or more of a clock, data, sync, and/or ground signal. The data channels 92, 94 may thereby each comprise a serial data bus such as, but not limited to, a Serial Peripheral Interface (SPI), Inter-Integrated Circuit (I²C), UNI/O, 1-Wire, or any other suitable data bus. The data channels 92, 94 may also be provided via a single shared data bus, in which case both data channels may be coupled to the processor 66 through a single data port. The first data channel 92 may couple the memory 80 to the console connector 82 so that the memory 80 is operatively coupled to the I/O interface 70 when the console connector 82 engages the connection point 32. The second data channel 94 may similarly couple a connection point in connector 82 to a matching connection point in the RTC connector 84. The second data channel 94 may thereby allow RTC 20 to be coupled to I/O interface 70 by treatment head 14 when console connector 82 engages connection point 32. In an alternative embodiment of the invention, the first and second data channels 92, 94 may comprise a single addressable data bus that is shared by the memory 80 of treatment head 14 and the RTC 20. For example, the data channels 92, 94 may be provided via a high speed serial Low Voltage Differential Signaling (LVDS) bus. Chipsets and/or circuits that support LVDS bus systems include the Serdes line of interface products, which are available from Texas Instruments Inc. of Dallas Tex., United States.

The treatment head 14 may also include passive circuits (not shown) such as inductors, capacitors, and/or resistors to filter or otherwise alter the signal transmission characteristics of the transmission line 86. These signal transmission characteristics may be modeled as a two-port network that connects the ultrasound signal ports 98, 100 with an impedance Z_{T} 102. The impedance Z_{T} 102 may be described in a numeric form by an impedance parameter data structure 104 stored in memory 80, and may be defined by a 2×2 matrix having four complex number values.

The RTC 20 includes the transducer 96, a memory 106, and a connector 108 configured to engage the RTC connector 84. The transducer 96 may be comprised of one or more transducer elements (not shown), and may be located in a sealed ultrasound chamber 110 filled with an acoustic medium 112 that couples the transducer 96 to an acoustic window 114. The acoustic medium 112 may facilitate the conduction of heat out of the transducer 96, as well as improve the transfer of ultrasound energy from the transducer 96 through the acoustic window 114 and into the patient. The acoustic medium 112 may be comprised of water or some other suitable liquid, gel, or solid material having desirable acoustic properties. The RCT 20 may also include an actuation assembly (not shown) that provides control over the position and/or orientation of the transducer 96 within the ultrasound chamber 110. An example of an RTC that includes an actuation assembly is described in U.S. Pub. No. 2012/0067750 filed on Sep. 15, 2011 and entitled “Modified Atmosphere Packaging for Ultrasound Transducer Cartridge”, the disclosure of which is incorporated herein by reference in its entirety.

Each of the one or more elements comprising transducer 96 (shown here as a single element for the purposes of clarity) may have an electrical input impedance Z_{XDCR} 116, which may be modeled as a single port impedance having a complex value. These input impedances may be stored as one or more data structures 118 in memory 106. In addition, the RTC 20 may be further characterized by electrical-to-acoustic and acoustic-to-electrical transfer functions. These transfer functions may include frequency dependent gain and loss terms that are also stored as data structures 118 in memory 106. Each of the one or more elements of transducer 96 may be coupled to an ultrasound signal port 120 of connector 108, with each port 120 being associated with an impedance Z_{XDCR} 116 and a set of transfer functions stored in memory 106 that characterize the electrical and acoustical properties of the RTC 20 with respect to that port 120.

In operation, each output port of the console 12, transmission line, or channel, provided by the treatment head 14, and element of transducer 96 in RTC 20 may be calibrated, and the resulting calibration parameters stored in the memory 68, 80, 106 corresponding to the associated component 12, 14, 20. These calibration parameters may then be retrieved from the associated memories 68, 80, 106 during operation of the system 10 and used by the controller application 73 to adjust the output power of the transmitter 40 and the signal measurements from the receiver 42. In this way, the controller 38 may compensate for inconsistencies in component performance when a new component is attached to the HIFU system 10, such as when the RTC 20 is replaced. The calibration parameters may also allow the controller 38 to compensate for non-idealities in the performance of a component over a range of power levels, temperatures, and frequencies. For example, in addition to impedance parameters, calibration parameters may include parameters that characterize system non-linearities, system gain variations, and the frequency response of system components. These calibration parameters may be determined as part of the component manufacturing process and stored in the memory 68, 80, 106 associated with the component in question. The stored calibration parameters may then be retrieved by the controller 38 and utilized to improve system performance by providing more accurate control and monitoring of the ultrasound energy delivered to the patient.

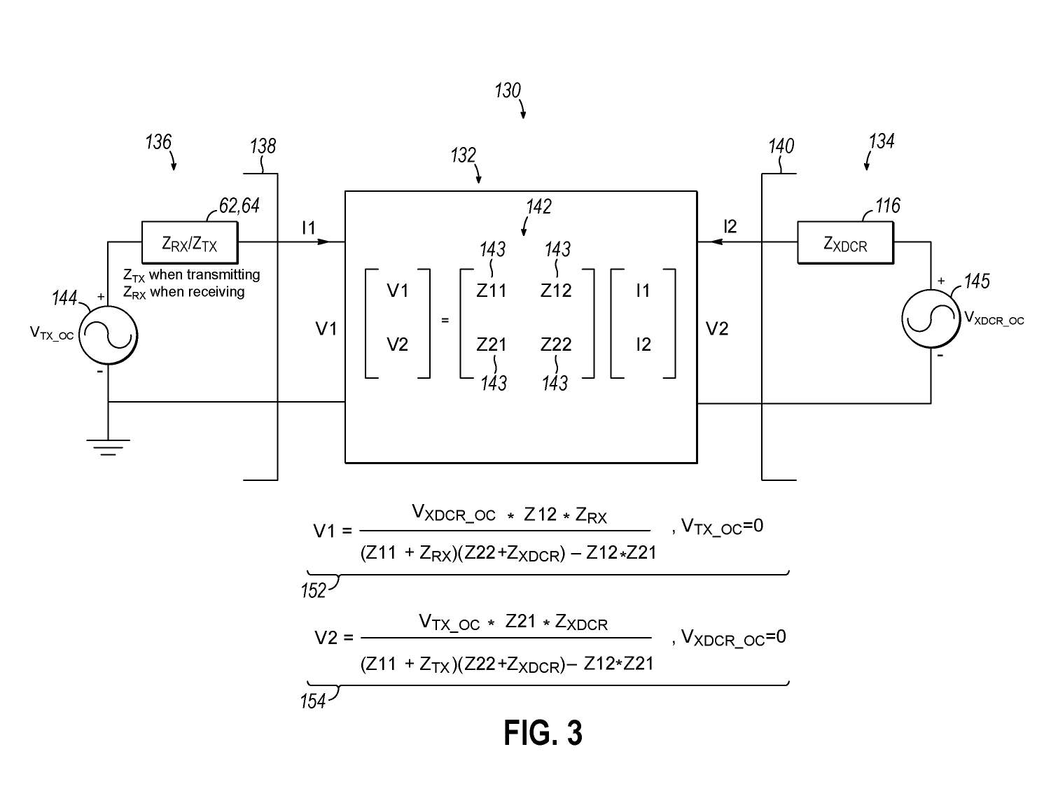

Referring now to FIG. 3, system components may be characterized by a signal transmission model 130 that includes a 2-port treatment head impedance model 132, a 1-port RTC impedance model 134, and a 1-port console impedance model 136. Each impedance, or Z-parameter, comprising the transmission model 130 may be a complex ratio of voltage to current that varies with frequency, and may be represented as a calibration parameter formatted as a complex number or phase vector. Although only a single system channel is illustrated in FIG. 3 for clarity, as discussed previously, the HIFU system 10 may include multiple channels, with each channel being represented by a separate signal transmission model 130. For example, in an embodiment of the invention, the console 12 may include 16 ultrasound transceivers 36 with each transceiver 36 associated with an independent set of Z-parameters. Likewise, there may be a plurality of treatment head impedance models 134, each representing a different transmission line 84, and a plurality of RTC impedance models 136 representing multiple elements of transducer 96. Embodiments of the invention are therefore not limited to an HIFU system 10 having any particular number of signal transmission models 130.

Signal transmission models 130 may be determined for a plurality of ultrasound signal frequencies spanning or exceeding the normal operating range of the HIFU system 10. For example, separate signal transmission models may be determined for each of four standard operating frequencies of the HIFU system 10, such as 1, 2, 3, and 3.75 MHz. Z-parameters for intervening frequencies may then be estimated using polynomial curve fitting to interpolate between the data points with a second or higher order polynomial. Because the Z-parameters typically have complex values, the polynomial interpolation may be performed independently for each of the real and imaginary values, or the magnitude and phase, depending on how the Z-parameters are formatted.

The HIFU system 10 may have multiple calibration points, or measurement planes, on the signal path between the ultrasound transceiver 36 and the transducer 96. For example, calibration planes 138, 140 may be defined at the connection points between the system components 12, 14, 20. The calibration planes 138, 140 may thereby allow the transmission characteristics of the channel coupling the ultrasound transceiver 36 and transducer 96 to be determined by the processor 66 using Z-parameter data stored as one or more data structures 74, 104, 118 in an associated component memory 68, 80, 106. The processor 66 may thereby adjust ultrasound output power levels and received signal measurements to compensate for performance variations in components attached to the console 12. Embodiments of the invention may thereby use the Z-parameters as calibration parameters to improve both power control and system monitoring accuracy as compared to systems lacking this feature.

The treatment head impedance model 132 may include a 2×2 matrix 142 that defines the signal impedance characteristics of transmission line 86 between the console 12 and the RTC 20 at a particular frequency. The Z-parameters 143 comprising the matrix 142 may be determined by measuring the electrical characteristics of the treatment head 14 using standard 2-port measurement techniques, such as with an automated 2-port network analyzer coupled to the ultrasound signal ports 98, 100 of connectors 82, 84. The Z-parameters 143 may include the effects of passive components in the transmission path provided by the treatment head 14, such as in-line or parallel inductors or other filter components. The Z-parameters 143 may be stored in memory 80 of treatment head 20 as one or more data structures 104. The Z-parameters may thereby be obtained by the controller 38 through the data port 76 while the console connector 82 is coupled to the connection point 32 of console 12.

The RTC impedance model 134 includes the RTC input impedance Z_{XDCR} 116. The RTC input impedance Z_{XDCR} 116 will typically be dominated by the input impedance of the transducer 96, but may also include the effects of any parasitic impedances or passive components included in the RTC 20. The RTC input impedance Z_{XDCR} 116 may be determined by measuring the input impedance of the RTC 20 with an impedance analyzer or other similar piece of test equipment. Similarly as described above with respect to the treatment head 14, the impedance Z_{XDCR} 116 (i.e., the single port Z-parameter of RTC impedance model 134) may be stored in memory 106 of RTC 20 as one or more data structures 118. The impedance Z_{XDCR} 116 may thereby be obtained by the controller 38 through the data port 78 while the RTC 20 is coupled to the connection point 32 of console 12 by the treatment head 14.

The console impedance model 136 may be characterized by a set of calibration parameters that includes: 1) the output impedance Z_{TX} 62 of transmitter 40; 2) the input impedance Z_{RX} 64 of receiver 42; and 3) an open circuit ultrasound driver voltage (V_{TX}_{_{—}}_{OC}) 144, which may be related to a ultrasound driver gain (G_{TX}) between the HVS 56 output voltage rail and the output of driver 58.

The receiver input impedance Z_{RX} 64 will typically be subjected to relatively low signal power levels, and may be measured with an impedance analyzer as discussed above with respect to the RTU input impedance Z_{XDCR} 116. The gain of the ultrasound receiver 42 may be calibrated after measuring the receiver input impedance Z_{Rx} 64 by measuring the receiver gain (G_{RX}) at multiple frequencies across the operational frequency range of the HIFU system 10. These measurements may take into account the effect of Z_{RX} 64 at each frequency. G_{RX} may be measured by providing a signal having a known amplitude and source impedance to the ultrasound signal port 44 at a frequency for which a calibration is desired. This may be accomplished, for example, by providing an ultrasound signal through a calibrated treatment head cable 18 having known 2-port Z-parameters. Based on the known signal source impedance, the known 2-port Z-parameters of the cable 18, and an expected nominal receiver gain, an expected receiver output voltage may be determined. The receiver 42 may then be queried, such as by the processor 66 to obtain the receiver output voltage, and a gain correction value determined from the ratio of the expected nominal gain to the actual measured gain G_{RX} at the test frequency.

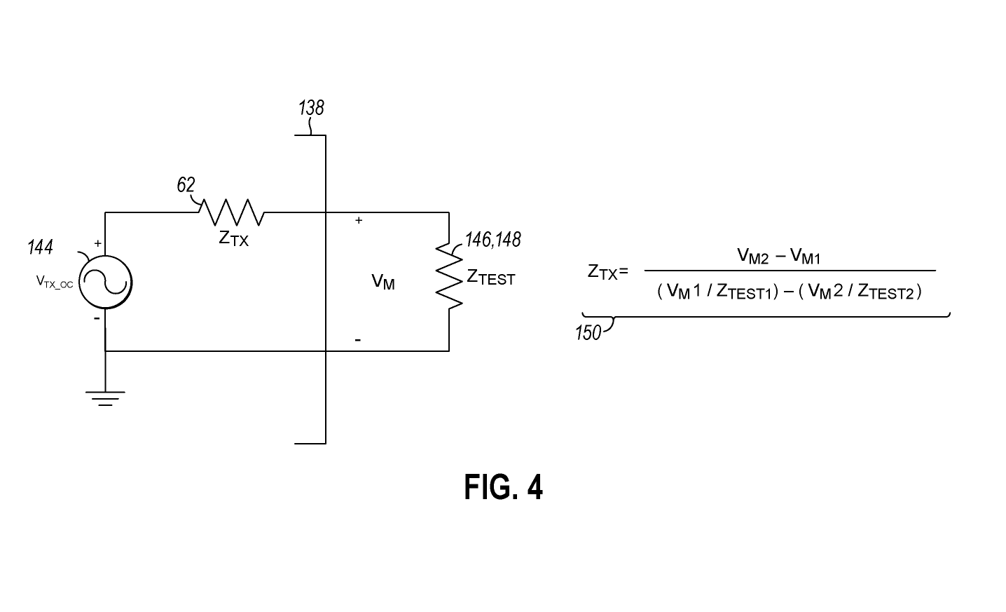

Because the ultrasound signal transmitter 40 typically operates at power levels that are too high for a conventional impedance analyzer, the transmitter output impedance Z_{TX} 62 may be determined using a procedure involving calibrated test loads. Referring now to FIG. 4, the complex system output impedance Z_{TX} 62 of ultrasound transmitter 40 may be determined using two test loads 146, 148 each having a different known impedance Z_{TEST1} and Z_{TEST2}. Each test load 146, 148 may be coupled to the signal port 44 in turn, and the resulting output voltages V_{M1} and V_{M2} measured. Based on the measured output voltages V_{M1}, V_{M2}, and the known test impedances Z_{TEST1}, Z_{TEST2}, the transmitter output impedance Z_{TX} 62 may then be determined by solving equation 150.

Once the transmitter output impedance Z_{TX} 62 is known, V_{TX}_{_{—}}_{OC} 144 may be determined Because it may be difficult provide an open circuit to the ultrasound signal port 44 at moderate to high frequencies, the open circuit voltage may be determined by coupling a known load such as Z_{TEST1} 146 to the ultrasound signal port 44. V_{TX}_{_{—}}_{OC} may then be determined by activating the ultrasound driver 58, measuring the resulting output voltage at the ultrasound signal port 44, and calculating an effective V_{TX}_{—}_{OC} based on the measured voltage, the value of the known load, and the known value of Z_{TX}. The G_{TX} of the ultrasound driver 58 may then be determined by comparing V_{TX}_{_{—}}_{OC} 144 to the DC voltage level of the HVS 56. G_{TX} may, in turn, be used to determine the HVS voltage levels required to achieve a desired V_{TX}_{_{—}}_{OC}. To allow the controller 38 to compensate for non-linearities in the transmitter 40, G_{TX} may be determined for a plurality of HVS voltage settings, e.g., three voltage settings. A line may be fitted to the points representing G_{TX} for each HVS voltage setting measured by connecting the two end points, by using linear regression, or by any other means suitable for modeling the output of the transmitter 40 over the range of measured HVS voltages. A voltage offset may then be determined at each HVS voltage setting that characterizes the difference between the measured output and the output predicted by the line for each drive level. These voltage offsets may allow the controller 38 to compensate for non-linearities in the output power of the transmitter 40 with respect to the supply voltage. As with the other console calibration parameters, the G_{TX} and voltage offset parameters may be stored as data structures 74 in memory 68 of controller 38.

In an embodiment of the invention having multiple ultrasound drivers 58, the output level control for each ultrasound driver 58 may be dependent on the voltage supplied by a single shared HVS 56. In systems sharing a single HVS 56, a common system level HVS output voltage may be calculated based on the individual channel calibration values for each of the active ultrasound signal transmitters 40. In another embodiment of the invention, the output voltage setting for the HVS 56 used to obtain G_{TX} may be a geometric mean of the expected maximum and minimum transmit power output levels of the HIFU system 10. In any case, the three console impedance model parameters Z_{RX}, Z_{TX}, and V_{TX}_{_{—}}_{OC}/G_{RX} for each channel may be stored as data structures 74 in memory 68 so that these calibration parameters may be accessed by controller applications 73.

Using the Z-parameters 143 of matrix 142 and the additional calibration parameters V_{TX}_{_{—}}_{OC} 144, Z_{XDCR} 116, Z_{TX} 62, and V_{XDCR}_{_{—}}_{OC} 145 to solve for V₁ and V₂ produces Equations 152 and 154. A relationship between the voltage V₁ at the console connector calibration plane 138 and the output of transducer 96 may be described by Equation 152. Similarly, a relationship between the RTC input voltage V₂ at the RTC connector calibration plane 140 and the output of the ultrasound driver 58 may be described by Equation 152. These Z-parameters may be stored in memories 68, 80, and 106 for use by controller applications 73 as calibration parameters to compensate for variations in component performance.

In an embodiment of the invention, an amount of electrical power P_{E} that must be delivered to the RTC 20 to generate a desired ultrasound acoustic output power at the patient may be determined based on an electrical to acoustic transfer function, or transducer scale/calibration factor (TSF). The TSF of RTC 20 may be determined by measuring acoustic energy output levels of the transducer 96 for one or more input voltages, and stored as one or more data structures in memory 106 of RTC 20. Based on P_{E} and the Z_{XDCR} 116 of transducer 96, the controller application 73 calculates the RTC input voltage V₂ required to generate the desired ultrasound acoustic output power at the patient. Working back from the RTC connector calibration plane 140, the controller application 73 can then determine the V_{TX}_{_{—}}_{OC} 144 required to generate the required RTC input voltage V₂ based on Equation 154. The accuracy of V_{TX}_{_{—}}_{OC} 144 may be further improved based on the values of G_{TX} and the voltage offset, which the controller application 73 may receive from memory 68 and use to determine an optimal HVS voltage setting to generate the required V_{TX}_{_{—}}_{OC} 144.

Controller applications 73 may thereby accurately control ultrasound power levels when new components are introduced into the HIFU system 10 without the need for system re-calibration. Although Z-parameters have been used herein as calibration parameters, persons having ordinary skill in the art will understand that any suitable parameter format may be used. For example, models may be constructed using S-parameters, Y-parameters, H-parameters, T-parameters, ABCD-parameters, or any other suitable parameters.

In addition to the Z-parameter models illustrated in FIGS. 3 and 4, other component performance characteristics may be obtained and stored as calibration parameters in one or more of the corresponding memories 68, 80, 106. In addition to compensating for impedance variations between components, the controller 38 may use calibration parameters to compensate for non-idealities in the performance of a component over a range of power levels, temperatures, and frequencies. For example, calibration parameters that characterize non-linearities in output power, system gain variations, transfer functions, and the frequency response of system components may be determined and stored in one or more of the memories 68, 80, 106. These calibration parameters may then be used to improve system performance during operation of the HIFU system 10. To this end, the calibration parameters may be retrieved from the associated memories 68, 80, 106 and used by the controller application 73 to adjust the output power of the transmitter 40 and to calibrate signal measurements from the receiver 42. In this way, the controller 38 may correct for differences in performance between components when components are exchanged as well as compensate for non-ideal component performance.

In an embodiment of the invention, the aforementioned TSF for the RTC 20 may be determined by measuring acoustic energy output levels of the transducer 96 for one or more input voltages and frequencies. These measurements may include processing input and output signals using a Fourier transformation to convert the signals into the frequency domain. The energy levels of the fundamental signal may thereby be isolated and compared so that the resulting transfer function characterizes transducer gain at the fundamental drive frequency. That is, by comparing the acoustic and electrical energy associated with the driven transducer in the frequency domain, the energy present in harmonics of the ultrasound signal may be ignored.

The TSF may be modeled as an ideal lossless transfer function and a loss term. The lossless transfer function may characterize the acoustic energy output for a given input energy, while the loss term may represent the energy lost in the conversion. Typically, the transfer function is reciprocal, and the loss term is not reciprocal. That is, the TSF may be symmetrical with respect to conversion of electrical energy into acoustic energy and conversion of acoustic energy into electrical energy, but not with respect to the energy losses associated with those conversions. Separate TSF values may therefore be characterized and stored in memory 106 for transmit and receive directions to account for differences in the loss and transfer functions at different power levels. For example, the transfer and loss functions may be different at the high power levels used to generate transmitted ultrasound as compared to the lower power levels associated with received ultrasound signals. The TSF parameters may be stored in memory 106 of the RTC 20 with the input impedance Z_{XDCR} 116 as one or more data structures 118. The TSF may thereby be obtained by the controller 38 through the data port 78 when the console connector 82 is coupled to the connection point 32 of console 12 in essentially the same manner as the impedance parameter Z_{XDCR} 116.

In an exemplary embodiment of the invention, the transducer impedance Z_{XDCR} 116 for each of a plurality of channels is measured at a frequency near the center of the operating range of the RTC 20. The transducer impedance Z_{XDCR} 116 is then stored in the memory 106 of RTC 20 and used by the controller 38 to adjust the output voltage of the HVS 56. The controller 38 may thereby cause the ultrasound driver 58 to generate an ultrasound signal that produces a voltage level at the transducer 96 sufficient to generate a desired Pulse Intensity Integral (PII) at the patient.

Once the transducer impedance Z_{XDCR} 116 is known, the TSF may be determined by providing a known amount of electrical energy to the input of the transducer 96 that would generate a desired acoustic energy output for an ideal transducer 96. The TSF may then be determined by measuring the acoustic output energy of the transducer and dividing the measured acoustic output energy by the known electrical energy provided to the transducer 96. The TSF may thereby provide an indication of the amount of electrical energy that must be provided to the transducer 96 to generate a desired amount of acoustic energy. If only a subset of the total number of transducer elements of the transducer 96 is being utilized, the TSF may be divided by the total number of transducer elements, and the resulting fractional TSF value used to determine the drive levels for each active channel. That is, for a sixteen element transducer, each element would be driven as if the element provides one sixteenth of the full TSF for the transducer 96. In an alternative embodiment of the invention, the TSF may be divided up by known, non-equal, fractional parts associated with each element. That is, different fractional adjustment values may be assigned to specific elements based on known characteristics of the element. In another alternative embodiment of the invention, a separate TSF may be determined for each element of the transducer 96, in which case the transducer element drive levels could be determined based on the TSF values for the active elements.

The amount of electrical energy provided to the transducer 96 under test may be determined by providing an ultrasound test signal having a known Root-Mean-Square (RMS) voltage to the transducer 96. The RMS voltage may be selected based on the transducer impedance Z_{XDCR} 116 so that a desired amount of real power is delivered to the transducer. That is, the test voltage is selected to deliver a desired amount of power to the real component of the complex impedance Z_{XDCR} 116. Because the response of the transducer 96 typically falls off rapidly outside the normal operating frequency range of the RTC 20, the TSF may be measured using energy generally confined to the center frequency of the test signal. To this end, the test signal voltage may be measured over a time period encompassing the active burst and any transients. The measured test signal may then be converted into the frequency domain by using a Fast Fourier Transform (FFT) scaled to provide Volts RMS. The FFT window may be made significantly wider than the burst duration in order to capture the tails of the test burst waveform. Taking into account the longer interrogation interval produces the following equation:

V_{RmsCenter}=V_{ScopeFFTideal}×Sqrt(SW/BL)

where:
V_{RmsCenter} represents the effective RMS voltage across the ideal burst length;
V_{ScopeFFTideal} represents the ideal scope reading at the frequency of interest;
SW represent the oscilloscope interrogation time, or the window length, which is typically about 20 uS; and
BL represents the ideal burst length over which the RMS voltage is to be measured, which may be for example 12.5 uS.

When using an FFT function on an oscilloscope, the peak voltage at the frequency of interest will typically not be representative of the true overall RMS voltage due to the window function applied by the oscilloscope. This window effect can be compensated for by simply applying a correction factor. For a rectangular window the equation above becomes:

V_{RmsCenter}=V_{ScopeFFT}×SW/BL

where :
V_{ScopeFFT} represents the actual reading from a typical scope at the frequency of interest.

Once the RMS voltage of the center frequency has been determined, the resulting RMS voltage may be squared and multiplied by the FFT window duration to yield a Voltage Squared Integral (VSI) value having units of V²-sec. The VSI value may then be divided by the transducer impedance Z_{XDCR} 116, with the real part of the quotient providing an Electrical Intensity Integral (EII) value having units of W-sec. To this end, the EII of the test burst waveform may be determined by:

EII=VSI/Z_{XDCR}

where:
Z_{XDCR} has a complex value, and
VSI represents the voltage squared integral of the test burst waveform. The real portion of EII may be determined by:

Re(EII)=Re(VSI/(|Z|Angleθ))=Re(VSI Angle(−θ)/|Z|)=VSI×Cos(θ)/|Z|;

and VSI may be determined by:

VSI=V_{RmsCenter}²×BL.

Using the formulae above for the oscilloscope FFT window factor we have:

VSI=V_{ScopeFFT}²×SW²/BL.

To determine the acoustic output energy, the PII may be obtained by measuring the output voltage of an acoustic test sensor that converts the acoustic pressure to an electrical voltage. Due to potential nonlinearities of the propagation medium, the full bandwidth of the signal may be used to determine the PII. This is in contrast to the EII determination discussed above, in which only energy at the center frequency of the test signal was used. A time based calculation that avoids performing an FFT may therefore be used to capture the energy in the full bandwidth of the measured ultrasound burst signal. The output voltage of the acoustic test sensor may be squared and integrated over the duration of the transmit burst to generate a VSI value for the burst. The VSI value may then be converted to Pressure Squared Integral (PSI) value having units of P²-sec by multiplying the VSI value by the pressure scale factor of the test sensor and taking into account the impedance of the medium. The PII in units of W-sec may then be determined from the PSI value by dividing the PSI value by the acoustic impedance of the acoustic medium between the RTC 20 and the acoustic test sensor. The TSF for the transducer 96 may then be determined from the ratio of the EII to PII. The TSF of the transducer 96 will typically be determined after installation in the final RTC assembly 20 so that any losses in acoustic and/or the electrical path of the RTC 20 are captured in the TSF.

Because a particular RTC 20 will normally only be operated at a specific frequency and ultrasound burst length, the TSF will typically be determined for the expected combination of signal frequency and burst length. However, embodiments of the invention are not limited to determining a TSF for a single frequency and/or burst type, and multiple TSF values may be determined and stored in memory 106 of RTC 20 for ultrasound bursts having different center frequencies and durations.

The persistence of vibration in the transducer after being excited by a short voltage pulse, or the “ring down” of the transducer 96 may also be calibrated. That is, ring down waveforms of the transducer 96 may be determined at the time of manufacture and compared to ring down limits specified by the transducer component specification. These limits may include minimum and maximum ring down durations, amplitudes, and/or frequencies. One or more ring down calibration parameters may then be determined by comparing the measured ring down characteristics for the specific transducer 96 to the specified limits. Calibration parameters that scale the specified ring down limits to the actual ring down characteristics measured at time of manufacture may then be determined and stored in memory 106. Typically, the transducer 96 includes an acoustic impedance matching layer that increases the operating bandwidth and efficiency of the transducer 96. One common failure mode occurs when this matching layer begins to delaminate, thereby altering the ring down characteristics of the transducer 96. In operation, the controller 38 may periodically measure the ring down of transducer 96 and check appropriate bounds as scaled by the calibration parameters stored in memory 118. Changes in the ring down characteristics of the RTC 20 as compared to the characteristics measured when new and unused may thereby provide an earlier warning of transducer failure as compared to merely using transducer manufacturer ring down specifications.

As described with respect to the ultrasound driver gain G_{TX} above, the HIFU system 10 may also include calibration parameters to allow the controller 38 to compensate for non-linearities in the output of the transmitter 40. To this end, the transmit gain and offset functions may be calibrated for linearity across the operating power output range of the HIFU system 10. Once the output impedance Z_{TX} 62 has been determined, the G_{TX} may be measured at a plurality of operational points (e.g., three points) within the operational power output range of the system 10. G_{TX} with respect to output power may then be modeled by line fitted to the measured operational points. A voltage offset may then be determined for each of the plurality of measured operational points based on the difference between the actual measured output and the G_{TX} value indicated by the fitted line. As with the transmitter output impedance values, G_{TX} and the corresponding offset voltages may be determined at multiple frequencies and stored in memory 68. The processor 66 may thereby improve output power control by compensating for variations in G_{TX} at different output power levels based on gain and offset data stored in memory 68. The determined values of G_{TX} will typically provide sufficient accuracy for small changes in ultrasound burst lengths about the nominal value used to generate the aforementioned calibration parameters. However, in alternative embodiments of the invention, G_{TX} and V_{TX}_{_{—}}_{OC} calibration parameters may be determined for multiple ultrasound burst lengths to determine additional offset and gain factors for adjusting the transmit output voltage V_{TX} across different burst lengths.

In operation, the controller 38 may use calibration parameters to improve the accuracy of power monitoring, thereby reducing the tolerances in the amount of ultrasound energy delivered to the patient. By increasing the accuracy of ultrasound energy delivery, the calibration factors may allow the HIFU system 10 to generate ultrasound energy levels that provide greater therapeutic benefits without harming non-targeted tissue. In an embodiment of the invention, the ultrasound driver 58 may drive the output voltage by alternately coupling the transmitter output 50 to the positive and negative output voltage rails of the HVS 56. The ultrasound driver 58 may thereby produce an output resembling a square wave having minimum and maximum voltage levels that are closely related to the rail voltages of the HVS 56. The acoustic power output may therefore be set by adjusting the voltage levels produced by the HVS 56. So that the electrical power being supplied by the HVS 56 can be determined, the HVS monitor circuit 59 may provide a signal to the processor 66 related to the current flowing into the HVS 56. The HVS monitor circuit 59 may be calibrated at the factory to improve accuracy.

The power monitoring function may be included in the controller application 73, and may determine a voltage value for the ultrasound burst signal delivered to each active transducer element in the RTC 20. This voltage value may be determined by determining the AC voltage being delivered to the transducer 96 based on the voltage of the HVS 56 and the calibration parameter data structures 74, 104, 118 stored in memories 68, 80, 106. The AC voltage may then be squared and integrated over a period encompassing the ultrasound burst window to produce a Voltage Squared Integral (VSI) value having units of V²-sec. In an alternative embodiment of the invention, one or more voltage sensors may be coupled to a connection point between the console 12 and the RTC 20. These voltage sensors may be configured to monitor the ultrasound signal voltage during operation of system, and thereby provide a voltage value for the ultrasound burst signal.

The HIFU system 10 may provide users with a plurality of selectable HVS voltage settings, and the HVS monitor circuit 59 may be calibrated for each of these settings. To this end, an offset calibration current in the HVS 56 may be determined for each voltage setting. In response to a change in the HVS voltage setting, the controller 38 may wait for a sufficient time to allow the HVS voltage to stabilize. Once the voltage has stabilized, an idle current reading may be obtained from the HVS monitor circuit 59 with the one or more ultrasound drivers 58 disabled. This idle current reading may be averaged over a period of time and stored in the memory 68 of controller 38 as an offset calibration value. The offset calibration value may then be subtracted from the HVS current readings while the one or more ultrasound drivers 58 are activated. The resulting current difference value may be averaged and used to represent an ultrasound signal current I_{OUT} being provided to the RTC 20. The harmonics of the ultrasound burst signal—which is generally a square wave at the output of the ultrasound driver 58—may be attenuated by the frequency response of transducer 96 and the passive components in Treatment Head 16. That is, some of the current provided to the ultrasound driver 58 that goes into generating ultrasound harmonics and is lost between the output of the diver 58 and the transducer 96. To correct for this loss, a correction factor may be applied to I_{OUT} to compensate for the difference between the current levels measured by the HVS monitor circuit 59 at the ultrasound driver 58 and those provided to the transducer 96. In an embodiment of the invention, this correction factor is about 0.9, and represents the difference between the average absolute value of the current provided detected by the HVS monitor circuit 59 and current provided to the transducer 96.

Ultrasound signal voltage and current determinations may be performed on individual channels in systems having a plurality of channels. However, in systems having an HVS 56 that is common to a plurality of ultrasound drivers 58, currents and voltages may be collectively determined for the active ultrasound drivers 58 sharing the HVS 56. In cases where the HVS 56 is shared by more than one ultrasound driver 58, the transmit voltages delivered to the transducer 96 by each channel may be determined using the channel calibration parameters to determine an overall average transmission calibration factor. This average transmission calibration factor may be calculated based on the number of active channels and their calibration parameters, and used to adjust the output power of the HIFU system 10 as well as to monitor system power output limits.

The power output of the system may be determined by squaring I_{OUT} and multiplying the squared output current by VSI to produce an energy squared output E² of system. The monitor limits applied to E² may be adjusted based on the calibration parameters stored in the memories 68, 80, 106 so that the E² limits accurately reflect the actual energy expected to be delivered by the system. This adjustment may result from the calibration parameters being applied to ultrasound signal voltages, component transfer functions, and/or to compensate for non-linearities in the system as described above. Embodiments of the invention may thereby provide improved monitoring of ultrasound power levels as compared to HIFU systems that lack calibration parameter storage features.

It will be understood that when an element is described as being “connected” or “coupled” to or with another element, it can be directly connected or coupled to the other element or, instead, one or more intervening elements may be present. In contrast, when an element is described as being “directly connected” or “directly coupled” to another element, there are no intervening elements present. When an element is described as being “indirectly connected” or “indirectly coupled” to another element, there is at least one intervening element present.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “in response to” means “in reaction to” and/or “after” a first event. Thus, a second event occurring “in response to” a first event may occur immediately after the first event, or may include a time lag that occur between the first event and the second event. In addition, the second event may be caused by the first event, or may merely occur after the first event without any causal connection.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method of controlling the acoustic power output of an ultrasound treatment system, the method comprising:
retrieving a calibration parameter of a component removably coupled to the ultrasound treatment system from a memory associated with the component; and
adjusting an output signal of an ultrasound signal transmitter based at least in part on the calibration parameter of the component.

2. The method of claim 1 further comprising:
determining the calibration parameter of the component; and
storing the calibration parameter in the memory associated with the component.

3. The method of claim 1 wherein the component is an ultrasound treatment head that includes a plurality of channels, and the calibration parameter includes a separate 2-port network model for each of the plurality of channels.

4. The method of claim 1 wherein the component is a replaceable treatment cartridge and the calibration parameter includes an electrical input impedance of the replaceable treatment cartridge.

5. The method of claim 1 wherein the component is a replaceable treatment cartridge and the calibration parameter includes an electrical-to-acoustic transfer function of the replaceable treatment cartridge.

6. A replaceable treatment cartridge for an ultrasound treatment system, the replaceable treatment cartridge comprising:
an acoustic transducer; and
a memory configured to store data relating to a calibration parameter of the replaceable treatment cartridge.

7. The replaceable treatment cartridge of claim 6 further comprising:
a chamber containing an ultrasound coupling medium configured to acoustically couple the transducer to a patient.

8. The replaceable treatment cartridge of claim 7 further comprising:
an electrical signal port coupled to the acoustic transducer,
wherein the calibration parameter includes an electrical-to-acoustic transfer function relating to an amount of ultrasound energy provided to the patient by the replaceable treatment cartridge in response to an ultrasound transmission signal at the electrical signal port.

9. The replaceable treatment cartridge of claim 7 further comprising:
an electrical signal port coupled to the acoustic transducer; and
an acoustic transmission window coupled to the ultrasound coupling medium,
wherein the calibration parameter includes an acoustic-to-electrical transfer function relating to an amount of electrical energy generated by the replaceable treatment cartridge at the electrical signal port in response to ultrasound energy incident on the acoustic transmission window.

10. The replaceable treatment cartridge of claim 6 wherein the calibration parameter includes a ring-down parameter of the acoustic transducer.

11. The replaceable treatment cartridge of claim 6 wherein the calibration parameter includes an electrical input impedance of the replaceable treatment cartridge.

12. The replaceable treatment cartridge of claim 6 further comprising:
a data port coupled to the memory and configured to transmit the data stored in the memory in response to the replaceable treatment cartridge being connected to the ultrasound treatment system.

13. The replaceable treatment cartridge of claim 6 further comprising:
a data port coupled to the memory and configured to transmit the data stored in the memory in response to receiving a query signal from the ultrasound treatment system.

14. The replaceable treatment cartridge of claim 6 wherein the acoustic transducer includes a plurality of transducer elements.

15. The replaceable treatment cartridge of claim 14 wherein the data relating to a calibration parameter of the replaceable treatment cartridge includes data relating to a calibration parameter of each of the plurality of transducer elements.

16. An ultrasound treatment system comprising:
an ultrasound signal transmitter;
a signal port coupled to the ultrasound signal transmitter and configured to accept a treatment head; and
a processor configured to obtain data relating to a calibration parameter of a component of the ultrasound treatment system, and to determine an output level of the ultrasound signal transmitter based at least in part on the data relating to the calibration parameter of the component.

17. The ultrasound treatment system of claim 16 further comprising:
a treatment head configured to accept a replaceable treatment cartridge, the treatment head including a cable coupleable to the signal port and a memory that stores data relating to a calibration parameter of the treatment head.

18. The ultrasound treatment system of claim 16 wherein the ultrasound signal transmitter includes a plurality of signal transmitter circuits and further comprising:
a treatment head including a connector configured to accept a replaceable treatment cartridge, a cable coupleable to the signal port, and a memory that stores data relating to a calibration parameter of the treatment head, the treatment head being configured to couple each of the plurality of signal transmitter circuits to the connector through a separate channel.

19. The ultrasound treatment system of claim 18 wherein the data relating to a calibration parameter of the treatment head includes data defining a plurality of 2-port network models, each of the 2-port network models being associated with one of the channels coupling the plurality of signal transmitter circuits to the replacement cartridge connector.

20. The ultrasound treatment system of claim 16 further comprising:
an ultrasound signal receiver having an electrical input impedance and coupled to the signal port,
wherein the processor is further configured to obtain data relating to the electrical input impedance of the receiver and to determine a level of the received input signal based at least in part on the electrical input impedance of the receiver and the calibration parameter of the ultrasound treatment system component.
